(19) United States
(12) Patent Application Publication
(10) Pub. No.: US 2010/0129477 A1
(43) Pub. Date: May 27, 2010

(54) COMPOSITION FOR TREATMENT OF SKIN DISORDERS

(76) Inventor: Zvi Ben Moshe, Rechovot (IL)

Correspondence Address: YORAM TSIVION, P.O. BOX 1307, PARDES HANNA, 37111, IL

(21) Appl. No.: 11/815,772

(22) PCT Filed: Feb. 6, 2006
(86) PCT No.: PCT/IL06/00144

§ 371 Date: Aug. 8, 2007

Related U.S. Application Data
(60) Provisional application No. 60/651,108, filed on Feb. 8, 2005.

(51) Int. Cl. A61K 31/375; A61P 17/06; A61K 36/752

(52) U.S. Cl. 424/736; 514/474

(57) ABSTRACT

A preparation for treating psoriasis vulgaris containing ascorbic acids and salts thereof, tartaric acid and vinegar, water and a carrier. A more potent preparation contains in addition vitamin B2 and several plant extracts.

### FIELD OF THE INVENTION

The present invention is in the field of skin care. The composition of the invention is preferably used for treating skin predicaments.

### BACKGROUND OF THE INVENTION

Psoriasis is a relatively common chronic disease associated with inflammation of the skin, causing also enhanced shedding of the skin. Psoriasis is not passed from person to person but is probably genetically linked. Although psoriasis is incurable, it can be managed to varying degrees by applying one or more treatment schemes.

Treatment methods for psoriasis are diverse. Topical treatments include, among others, the application of emollients, vitamin D or derivatives thereof (calcipotriol), and cortisone. Cortisone may also be injected in specific locations in which the disease is especially persistent. Another type of topical treatment utilizes retinoids which are related to vitamin A. Such may be applied locally alone or in combination with cortisone.

Systemic treatments used for psoriasis are drugs that affect the whole body. For example cyclosporine that is used in cases of widespread psoriasis. This treatment is potentially harmful and must be monitored carefully.

Phototherapy is a method of treatment exploiting light. Especially UV light at various wavelengths and combination thereof is used.

### DETAILED DESCRIPTION OF THE PRESENT INVENTION

In accordance with the present invention, compositions for treating dermatological disorders are prepared, containing several active components. The application of the preparations in accordance with the embodiments of the invention to skin of patients provide a relief to symptoms of psoriasis to some extent. A principal component of the composition of invention is vitamin C, also known in its chemical name L-ascorbic acid. The plain acid is used as a component, as well as salts thereof, typically sodium and calcium ascorbate. Two more components are mixed together with the vitamin C in a basic preparation of the invention. One is tartaric acid, and the other one is vinegar. Vinegar is a fermentation product of plant extracts, juices or starch, containing dilute acetic acid and some other organic acids. A typical preparation in accordance with an embodiment of the invention contains also a carrier. A preferred carrier used is glycerine. Water is used as a common solvent, for solubilising and help mixing all the ingredients homogeneously together. A formula of the fundamental preparation of the invention is given in Table 1, in weight per weight percentages.

TABLE 1

| Component | Percentage w/w |
|---|---|
| Ascorbic acid and | 10-25 |
| ascorbic acid salts | |
| Tartaric acid | 0.2-4 |
| vinegar | 1-10 |
| Glycerine | 20-50 |
| Water | 30-60 |

For obtaining a more potent and a faster reacting preparation, several more components are added, to form a preparation type referred to hereinafter as advanced preparation. The additional components are classified as follows: plant extracts and vitamin B2 (riboflavin). The plant extracts used in an advanced preparation are lemon juice, extracts of Taraxacum officinale (dandelion), Opuntia coccinellifera (a cactus) fruit extract, Cereus grandiflorus (a cactus) young stems and flowers extract. A full description of the advanced preparation in weight per weight percentages is in given in Table 2.

TABLE 2

| Component | Percentage w/w |
|---|---|
| Ascorbic acid and | 10-25 |
| ascorbic acid salts | |
| Tartaric acid | 0.2-4 |
| vinegar | 1-10 |
| Vitamin B2 | 0.05-0.2 |
| Cereus grandiflorus | 0.5-2 |
| Opuntia coccinellifera | 0.5-2 |
| Lemon Juice | 0.5-4 |
| Taraxacum extract | 1-10 |
| Glycerine | 100 ml |
| Water | 20-50 |

The preparations of the invention can be used as paste or ointment, for applying to the skin of the patient.

1. A composition for treating psoriasis vulgaris comprising:
a mixture of components comprising:
ascorbic acid and salts of said acid, at 10-25%;
tartaric acid, at 0.2-4%;
vinegar, at 1-10%;
water, at 30-60, and
a carrier; at 20-50%,
and wherein said composition is used as paste and ointment.

2. A composition for treating psoriasis vulgaris as in claim 1 further comprising:
vitamin B2, at 0.05-0.2%;
lemon juice, at 0.5-4%;
Cereus grandiflorus extract, at 0.5-2%;
Opuntia cocinellifera extract, at 0.5-2%, and
Taraxacum officinale extract, at 1-10%.

3. A composition for treating psoriasis vulgaris as in claim 1 wherein said carrier is glycerine.

4. A composition for treating psoriasis vulgaris as in claim 2 wherein said carrier is glycerine.
